(19) United States
(12) Patent Application Publication
(10) Pub. No.: US 2018/0271410 A1
(43) Pub. Date: Sep. 27, 2018

(54) SYSTEMS, METHODS, AND APPARATUSES FOR ACTIVITY MONITORING

(71) Applicants: Rajesh Rawal, Fullerton, CA (US); Sreenivasan Narayanan, Lincolnshire, IL (US)

(72) Inventors: Rajesh Rawal, Fullerton, CA (US); Sreenivasan Narayanan, Lincolnshire, IL (US)

(21) Appl. No.: 15/934,594

(22) Filed: Mar. 23, 2018

Related U.S. Application Data
(60) Provisional application No. 62/475,670, filed on Mar. 23, 2017.

(51) Int. Cl. A61B 5/11; A61B 5/00; G08B 21/04; H04W 4/021; H04W 4/12

(52) U.S. Cl. CPC A61B 5/1118; A61B 5/1117; A61B 5/1126; A61B 5/746; A61B 2562/0219; G08B 21/0446; G08B 21/043; H04W 4/021; H04W 4/12; G08B 21/0469

(57) ABSTRACT

Methods, systems and apparatuses, including computer programs encoded on computer storage media, are provided for monitoring a user's activity, orientation, and/or transitions between activities and/or orientations. A motion detection device is placed on a user's body in a manner to prevent the user from removing the device. The device detects the user's activities, orientations, and/or transitions via one or more accelerometers, gyroscopes, etc. The device transmits data regarding the activities, etc., to a server for logging and analysis. The data may be transmitted via a gateway device. Based on the analysis of the data, one or more notifications may be sent to, e.g., providers of the user. The motion detection device can detect that it has been removed from the user's body through a proximity detector, e.g., an infrared sensor. The motion detection device may include a button that can be pushed by a provider to indicate that a situation causing a notification, e.g., that the user has fallen, has been resolved.

### CROSS-REFERENCE TO RELATED APPLICATIONS

The present application claims benefit of U.S. provisional patent application Ser. No. 62/475,670, titled “Systems, Methods and Apparatuses for Activity Monitoring,” filed Mar. 23, 2017, which is incorporated by reference herein in its entirety.

### BACKGROUND

This specification relates generally to systems, methods, and apparatuses for monitoring the activity, location, and/or orientation of a user. Some of the present embodiments also relate generally to a system and method for determining when a user has moved outside a predefined area.

Dementia, which includes Alzheimer's disease, currently affects more than 45 million people worldwide. Symptoms of dementia include the loss of cognitive functioning, including thinking, remembering, and reasoning. As a result, people living with dementia may become disoriented and lost, even in places that are familiar to them, including their own neighborhood. This is known as wandering. If a wandering person is not found within 24 hours, up to half of individuals who wander may suffer serious injury or death.

People suffering from dementia are also at high risk of falling. They are more than three times as likely to fracture their hip when they fall, which may lead to surgery and immobility. The rate of death following a hip fracture for those with dementia is also increased.

Therefore, there is a need for systems and methods for monitoring the movements of a user, so it can be detected when the user has fallen or performed other actions and/or when the user has moved outside a predefined area.

### SUMMARY

In accordance with the foregoing objectives and others, methods, systems, and apparatuses are disclosed herein that allow for monitoring the motions, movements, and/or orientations of a user, including standing, sitting, lying down, running, walking, and/or falling. Methods, systems, and apparatuses are also disclosed herein that allow for monitoring the location of a user.

In one embodiment, a device is provided that can be worn by the user or otherwise placed on the user's person. The device may include a proximity sensor to detect when the device has been removed (i.e., is no longer being worn) by the user.

The device may also include one or more sensors to detect motion and/or orientation, such as accelerometers, magnetometers, gyroscopes, and/or compasses. The motion and/or orientation detected by the sensor(s) is analyzed, and a signal indicating the type of activity being performed by the user (e.g., being still, sitting, standing, walking, running, falling, etc.) may be sent to a server for logging at predefined periodic intervals. Activity transition points, e.g., when the user moves from sitting to standing, may also be sent to the server. The activity information and activity transition point may be stored in the device, displayed on the device, and/or otherwise communication to the user, e.g., through one or more notifications. In an alternative embodiment, the raw sensor data may be sent to the server for analysis.

In one embodiment, one or more notifications may be sent to a user and/or provider when the user has moved outside of a predefined area, e.g., the device is out of communication range of a gateway. Additionally or alternatively, one or more notifications may be sent to a provider when certain activities and/or transition points are detected, e.g., when the user has fallen. The device may comprise a button or other means for the provider to indicate that the situation has been resolved, e.g., the wandering user has been located or the fallen user has been assisted. The device may be configured to be placed on the user's back, to prevent the user from pressing or otherwise activating the button or other means for indicating.

In one embodiment, one or more notifications may be provided to a user and/or provider relating to aggregate movement statistics, e.g., how often the user has fallen, how often the user has moved outside the predefined area, how much time the user has spent walking, etc. Activity monitoring criteria (e.g., what activities are monitored, length of the recording interval, etc.), notifications relating to specific activities, and other settings may be set by a user and/or by a provider associated with the user via one or more software applications.

One aspect of the present disclosure comprises a device that attaches to a user's body, comprising a location detector; a motion detector; an orientation detector; a communication module for transmitting user activity information to a server based on activity monitoring criteria, wherein the user activity information causes the server to transmit periodic notifications to a provider; a proximity detector for detecting that the device has been removed from the user's body; and means for causing the server to stop transmitting the notifications.

Implementations include: the user activity information comprises the user's location and the notifications are transmitted based on the user having left a predefined geographic area; the user activity information comprises the user's motion, and the notifications are transmitted based on the user falling or haven fallen; the proximity detector comprises an infrared detector; the communication module transmits the user activity information to a gateway and the gateway transmits the user activity information to the server;

Another aspect of the present disclosure comprises a method for notifying a provider that a user possessing a motion detection device needs assistance, comprising: receiving, by a server, activity information regarding the user from the motion detection device, the activity information based on activity monitoring criteria previously transmitted to the motion detection device; determining, by the server, that the user needs assistance based on the received activity information; and sending, by the server, periodic notifications to the provider regarding the assistance needs of the user; wherein the motion detection device comprises a button that may be pressed by the provider causing the server to stop sending the notifications.

Implementations include: the user activity information comprises the user's location and the notifications are transmitted based on the user having left a predefined geographic area; wherein the user activity information comprises the user's motion, and the notifications are transmitted based on the user falling or haven fallen; the motion detection device comprises a proximity detector for detecting that the device has been removed from the user's body; the proximity detector comprises an infrared sensor; the server receives the user activity information from a gateway, wherein the gateway had previously received the user activity information from the motion detection device.

Another aspect of the present disclosure comprises a system comprising one or more processors and one or more storage devices storing instructions that when executed by the one or more processors cause the one or more processors to perform operations comprising: receiving, from a motion detection device, activity information regarding a user possessing the motion detection device, the activity information based on activity monitoring criteria previously transmitted by the system to the motion detection device; determining that the user needs assistance based on the received activity information; and sending periodic notifications to a provider regarding the assistance needs of the user; wherein the motion detection device comprises a button that may be pressed by the provider causing the server to stop sending the notifications.

Implementations include: the user activity information comprises the user's location and the notifications are transmitted based on the user having left a predefined geographic area; the user activity information comprises the user's motion, and the notifications are transmitted based on the user falling or haven fallen; the motion detection device comprises a proximity detector for detecting that the device has been removed from the user's body; the proximity detector comprises an infrared sensor; the system receives the user activity information from a gateway, wherein the gateway had previously received the user activity information from the motion detection device.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows one embodiment of a system according to the principles of the present disclosure.

FIG. 2 shows one embodiment of a motion detection device according to the principles of the present disclosure.

FIG. 3 shows one embodiment of a gateway according to the principles of the present disclosure.

FIG. 4 shows one embodiment of a server according to the principles of the present disclosure.

FIG. 5 shows a method for entering activity monitoring criteria and notification preferences according to the principles of the present disclosure.

FIG. 6 shows a method for analyzing activity information and sending a notification according to the principles of the present disclosure.

### DETAILED DESCRIPTION

Various methods, systems, and apparatuses are discussed herein that monitor the activity of the user. The described embodiments allow for the notification of users and/or providers regarding the user's activities, the collection of information relating to the user's activities, and/or the reporting of such information to the user and/or a provider associated with the user.

The disclosed embodiments comprise a motion detection device that is placed on the user's person. The disclosed embodiments may further comprise a gateway device in communication with the motion detection device. The gateway may also be in communication with a server through a network. The server may store user information, including notification information and/or activity information, in a database. This information may be accessible via an application running on a provider device and/or a user device. This application may be in the form of a downloadable application installable and executable on a device such as a computer, smartphone, or tablet. Additionally or alternatively, the application may be available as a web application, accessible via a client device having an internet browser.

As used herein, “user information” includes, but is not limited to: user identification information (e.g., name, age, date of birth, sex, social security number, unique ID, photo, etc.); contact information (e.g., email address, physical address, phone number, etc.); and/or billing information (e.g., credit card information, billing address, etc.). User information may also include information relating to one or more motion detection devices associated with the user (e.g., MAC address, unique device ID, etc.); notification preferences; and/or information relating to third-parties associated with the user (e.g., a doctor or attending or other health care provider who may be sent notifications).

As used herein, “activity information” includes, but is not limited to: type of activity (being still, walking, running, falling, etc.); user orientation (e.g., lying down, sitting, standing, etc.); start and stop times of activities; activity and orientation transition times; times of falls; times of wandering; etc.

As used herein, “activity monitoring criteria” includes, but is not limited to: the types of activities and/or orientations to be sent to the server (e.g., being still, sitting, standing, lying down, walking, running, falling, etc.), frequency of sending activity information to the server, which activity transitions (e.g., being still to walking, sitting to standing, etc.) are to be sent to the server, etc.

As used herein, “notification preferences” include, but are not limited to: the destination(s) (e.g., email address(es), phone number(s) (for SMS, etc.), motion detection devices, provider devices, etc.) of the notification; the type of notification (e.g., audio alert, visual alert, SMS, email, push notification of an app running on a provider device, etc.); notification content for SMS, email, and push notifications (e.g., a message that says “User A has fallen!”); the volume of an audio notification (including mute, or no sound); the number of times the audio notification sounds; the frequency of the audio notification (if the audio notification is a single tone or series of tones); a multi-note audio notification selection; the brightness and/or color of a visual notification; the number of LED flashes if the visual notification is an LED; the number of separate notification windows (e.g., times the notification will be presented); the time between notification windows; the trigger for the notification (e.g., a particular activity that causes the notification to be sent, etc.); etc.

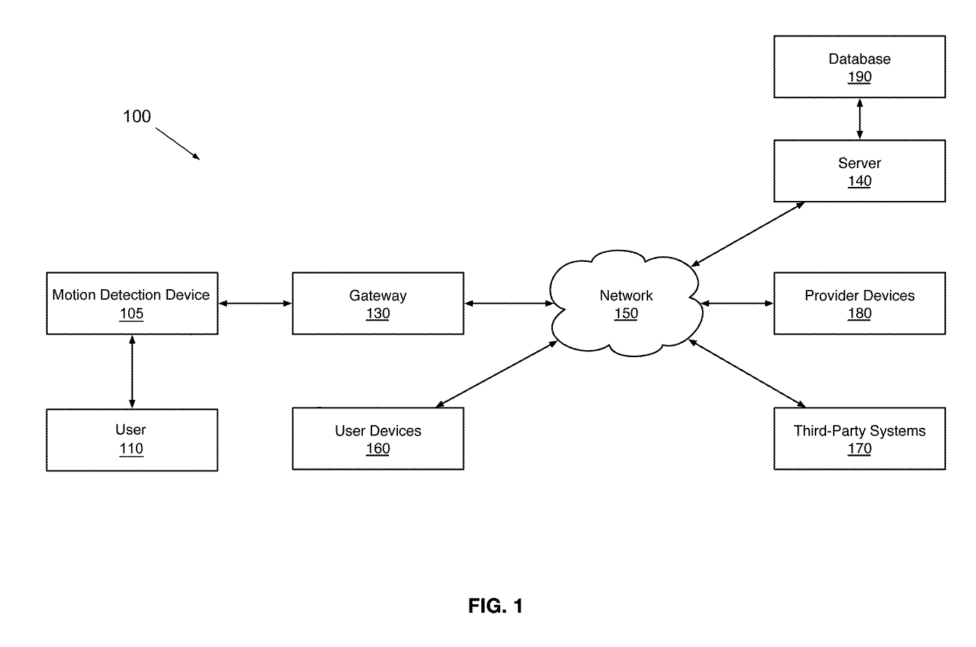

Referring to FIG. 1, an exemplary system 100 is presented. As shown, the system comprises a motion detection device 105 carried by user 110. The motion detection device 105 may be in communication with a gateway 130, via a wireless connection, e.g., a Bluetooth Low Energy (BLE) connection. The system may also comprise a server 140 in communication with a database 190, which stores user information, including notification preferences and/or activity information. The gateway and the server may be in communication via a network 150 (e.g., Internet, LAN, cellular, intranet, etc.). While one motion detection device 105 is shown, multiple motion detection devices may be in communication with the server (e.g., via one or more gateways 130).

The motion detection device(s) 105 are adapted to detect certain user activities, such as when the user is being still, walking, running, falling, etc., and/or user orientations, such as lying down, sitting, standing, etc. When a motion detection device detects such an activity, information regarding the activity is stored in the device and/or transmitted to the gateway 130 (e.g., via Bluetooth Low Energy (BLE), NFC, RFID, ZIGBEE, WiFi, or cellular link (e.g., CDMA or GSM)).

The gateway 130 is adapted to transmit activity information received from the motion detection device 105 to the server 140 via the network 150. The gateway may be further adapted to receive notification preferences from the server 140 via the network 150 and transmit the received notification preferences to the motion detection device 105.

The server 140 may be adapted to receive, determine, record, and/or transmit user information for any number of users. Such information may be manually entered or selected by a user via an online, mobile, or desktop application running on a user device 160 or a provider device 180. User information may additionally or alternatively be automatically received from a client device (e.g., a user device 160 or provider device 180), and/or any number of connected motion detection devices 105 (either directly or indirectly via an intermediate device). The server may store received or determined information in, for example, the database 190.

Notification preferences may also be stored in the database 190. The system is adapted to provide notifications to providers (e.g., via provider device(s) 180). Generally, the system is configured to automatically transmit notifications to providers based on predefined, rule-based events stored in the database 190. For example, the user's provider(s) may be notified when the transmitted activity information indicates that the user has fallen or that the user is outside the predefined area. The notification(s) may be in the form of a visual alert (e.g., LED or other light), an audio alert (e.g., via a speaker or buzzer), or a message (e.g., a push message, an SMS message, or email) sent to one or more provider device(s) 180. Details regarding the notification(s) are based on the notification preferences, which may be set up via a user device 160 or a provider device 180. In one embodiment, the notification(s) may be sent to the provider(s) on a periodic basis until a button on the motion detection device has been pressed by a provider, or the provider otherwise signals the server that the event leading to the notification has been resolved (e.g., the user has been located if wandering notification was sent, the user was cared for if a notification was sent indicating that the user had fallen or another activity occurred, etc.).

Optionally, the server 140 may be connected to one or more third party systems 170 via the network 150. Third party systems may store information in one or more databases that may be accessed by the server. Third party systems may include, but are not limited to: electronic medical record systems; other healthcare provider systems; calendaring and/or scheduling systems; backup systems; communication systems and/or others.

The server may be capable of retrieving and/or storing information from third party systems 170, with or without user interaction. Moreover, the server may be capable of communicating stored information to third party systems, and may notify users of such communications.

The system may include one or more provider devices 180. The provider devices allow providers for the user to receive notifications, e.g., a notification that the user has fallen. The provider devices also allow health providers or other parties to create, access, update and/or delete user information stored in the database 190, e.g., via a client application running on a provider device.

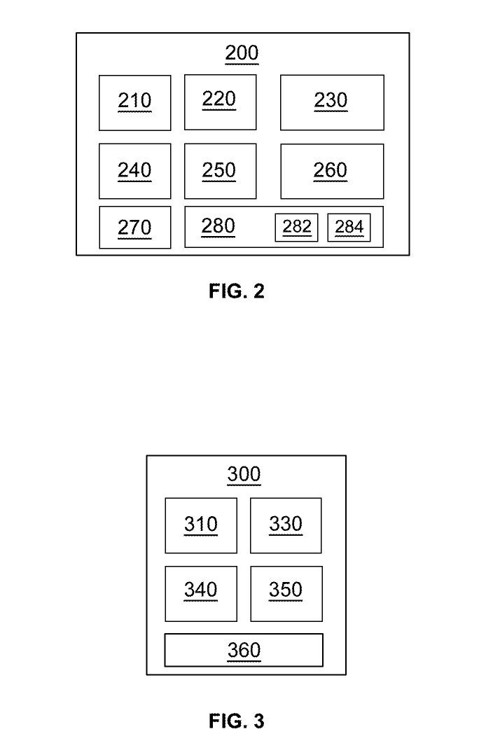

Referring to FIG. 2, a block diagram of an exemplary motion detection device 200 is illustrated. The device 200 comprises a communications module 210, a motion detection module 220, a proximity module 230, a processor 240, a power source 250, memory 260, a wandering detection module 270, and a notification module 280 (optionally comprising one or more audio transducers 282 (e.g., speakers, buzzers, etc.) and/or visual transducers 284 (e.g., LEDs, etc.)). The motion detection device further comprises a device ID, such as a MAC address or other unique ID, which may be employed to distinguish the device from any other devices that may also send or and/or receive messages to and/or from the server 140.

The motion detection module 220 may comprise one or more sensors or other devices that detect motion and/or orientation of the user. Any number of motion sensors, e.g., accelerometers, magnetometers, gyroscopes, compasses, etc., may be used to detect motion. In one embodiment, an all-in-one unit (e.g., InvenSense ICM-20648) may be used.

The motion detection module may be configured to detect a number of motions and/or orientations of the user. For example, the motion detection module may detect that the user is walking, running, falling, being still (e.g., no movement), sitting, standing, lying down, etc.

The communication module 210 may comprise a wireless communications circuit, such as BLE, NFC, RFID, ZIGBEE, WiFi, or cellular link (e.g., CDMA or GSM). The communication module may be employed to send and/or receive real-time or stored information, such as activity information and/or notification information, to and/or from the gateway 130 via BLE or other communications protocols.

In one embodiment, the communication module may be configured to send activity information to the gateway 130 according to the activity monitoring criteria stored in the motion detection device. For example, the motion detection device may be configured (e.g., via activity monitoring criteria) to send activity information regarding the user's current activity and orientation once every minute.

As another example, the motion detection device may be configured (e.g., via activity monitoring criteria) to send activity information when a sudden transition between standing and lying down is detected (possibly indicating a fall). If such a sudden transition is detected, activity information may be sent to the gateway indicating that the user has fallen.

The activity information may include the detected activity and/or orientation, the ID of the motion detection device, and the date and time. The activity information may then be forwarded to the server 140 by the gateway so the user's record can be updated.

In an alternative embodiment, the raw data from the motion and/or orientation sensors is sent to the server for analysis. In this embodiment, the raw data is sent at intervals defined by a sampling frequency, which is part of the activity monitoring criteria. In this embodiment, the raw data is analyzed by the server to determine if the user's activities trigger any notifications.

The proximity module 230 is configured to generate a message when the user is no longer wearing the motion detection device. The proximity module may detect that the user is no longer wearing the motion detection device via a sensor, e.g., an infrared sensor, to detect when the device is in proximity to the user's body. When the proximity module detects that the user has removed the device from the user's person or the device has fallen from the user, data may be sent to the server via the gateway indicating the removal of the device. A notification may then be sent to one or more providers, according to the notification preferences in the user's account.

The optional wandering detection module 270 is configured to generate a message when the user has exited a predefined area. In one embodiment, the wandering detection module comprises a GPS unit or other location detection device and includes a predefined area the user is allowed to be in. If the user exits the predefined area, according to a comparison of the user's present location and the predefined area, a message may be sent to the server via the gateway indicating that the user is wandering. A wandering notification may then be sent to one or more providers, according to the notification preferences in the user's account. The predefined area may be set by a provider at a provider device 180.

The motion detection device may further comprise a means to indicate that the situation leading to a notification has been resolved. In one embodiment, the means may comprise a button (physical or digital) for use by a provider to indicate that the user has been located (e.g., if wandering notification was sent), or otherwise cared for (e.g., if a notification was sent indicating that the user had fallen or another activity occurred). In one embodiment, the notification may be sent to the provider(s) on a periodic basis until the button or other means has been pressed. In the embodiment where the motion detection device is configured to be placed on the user's back, the user is prevented from pressing the button.

The notification module 280 may be configured to notify the user of one or more statuses, including removal of the motion detection device from the user's body, the user has exited the predefined area, etc. The notification can be in the form of an audio notification (e.g., a sound or music generated by a speaker or buzzer 282) or a visual notification (e.g., a flashing or non-flashing light, such as a LED light 284). The notification preferences may be specified by the user and/or the user's provider, and may be configurable at the motion detection device, the provider device, or the server.

The motion detection device further comprises a processor 240, such as a general purpose microprocessor, special purpose microprocessor, and/or any other kind of central processing unit (“CPU”). The motion detection device may also comprise memory 260 in communication with the processor, communications module 210, motion detection module 220, proximity module 230, and/or notification module 280, such as but not limited to, ROM (e.g., NAND flash, NOR flash, flash on another processor, other solid-state storage, mechanical or optical disks) and/or RAM. The memory may store any of the information described herein, such as but not limited to activity information, activity monitoring criteria, and/or notification information. Flash memory lends itself to applications such as the present in that it has good shock resistant characteristics and may retain stored data without the need for an active power source.

The memory may store executable code or instructions for one or more applications. When an application is requested to be executed, the processor retrieves corresponding executable code and/or data from the memory and executes it. The executable code can be temporarily or permanently stored on the memory or storage of the application processor.

The device may comprise one or more power sources 250 that can be utilized to power the electrical components thereof. In certain embodiments, the motion detection device may comprise a removable and/or rechargeable power source, such as a rechargeable battery. If a rechargable power source is included, the rechargeable power source may be recharged through wired or wireless (e.g., inductive) rechargers.

The motion detection device 200 is sized to be able to be placed on the user's person. In one embodiment, the device may measure less than 5 cm in length and height, e.g., about 3 cm, and less than 2 cm in width, e.g., about 1 cm. The device may be configured to be placed on the user's back using, e.g., a clip, to help prevent the user from making adjustments to the device and/or pressing the button or other means for the provider to indicate that the user has been located (e.g., if wandering notification was sent), or otherwise cared for (e.g., if a notification was sent indicating that the user had fallen or another activity occurred) has been resolved.

Referring to FIG. 3, a block diagram of an exemplary gateway 300 is illustrated. As shown, the gateway 300 may comprise a communications module 310, a wandering detection module 330, a processor 340, a power source 350, and a memory 360.

The communication module 310 enables communication with the motion detection device 105 and the server 140. The communication module comprises a wireless communications circuit, such as BLE, NFC, RFID, ZIGBEE, WiFi, or cellular link (e.g., CDMA or GSM). The communication module may be employed to send and receive real-time or stored information to and from the server 140 (e.g., via WiFi or cellular) and/or the motion detection device 105 (e.g., via BLE, Wifi, or other communications protocols).

The gateway, through the communication module, receives activity information from the motion detection device 105 and transmits such information to the server 140 for logging and/or analytics. The gateway also receives information, such as activity monitoring criteria and/or notification preferences, from the server 140 and transmits the information to the motion detection device 105.

The wandering detection module 330 is configured to generate a message when the user has exited a predefined area. In one embodiment, the wandering detection module detects when the communication link (e.g., BLE, Wifi, etc.) between the motion detection device and the gateway has been broken, e.g., because the motion detection device has moved outside the range of the gateway's communication module. If the communication link is broken, a message may be sent to the server indicating that the user is wandering. A wandering notification may then be sent to one or more providers, according to the notification preferences in the user's account.

The processor 340, power source 350 and memory 360 may be implemented in similar fashion to that described above with respect to the motion detection device.

In an alternative embodiment, the functionality of the gateway 300 may be incorporated into a user device 160. In this embodiment, the motion detection device 105 may be configured to communicate with the user device through a wireless protocol (e.g., Bluetooth or BLE) and the user device may be configured to communicate with the server 140 through a wireless protocol (e.g., WiFi or cellular). Using a user device, a user may view and/or update user information (e.g., activity monitoring criteria) associated with one or more motion detection devices in communication with the device (e.g., via a native application installed on the user device and/or via a web application accessed by the user device).

In a further alternative embodiment, where the motion detection device has sufficient power for uninterruptable communication over the network, the functionality of the gateway may be incorporated into the motion detection device. In this embodiment, the combined device sends and/or receives information to and/or from the server without the intermediate step of going through a gateway. In this embodiment, the motion detection device may be configurable from the server, a provider device, and/or a user device.

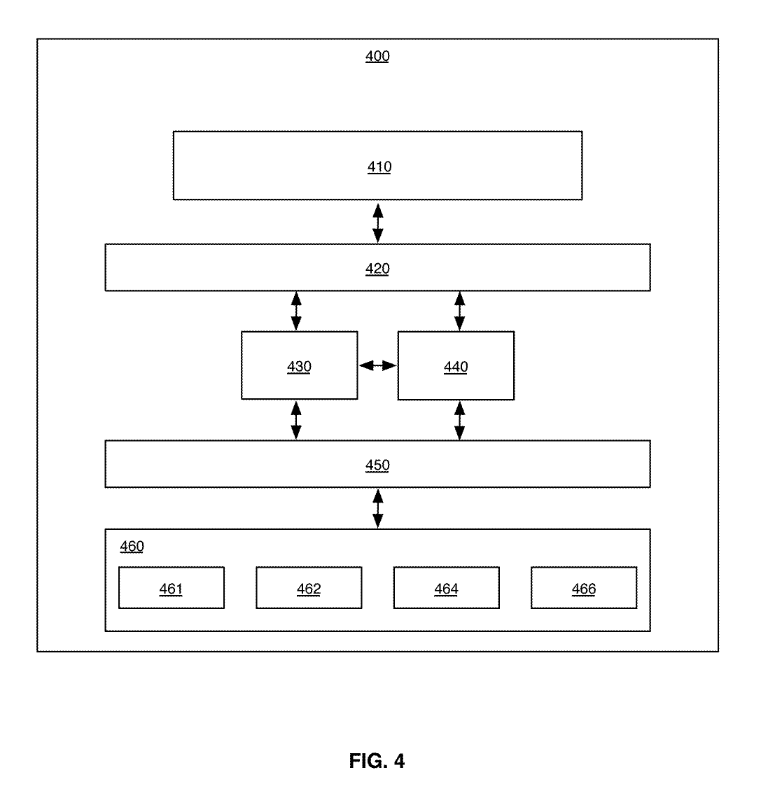

Referring to FIG. 4, a block diagram of an exemplary server 400 is illustrated. As shown, the server 400 may comprise hardware 460 such as any number of processors 462, RAM 464 and internal or external memory 466. The server may include a network interface 461 such that it may access a network to send or receive information.

As shown, at least one database 410 may be accessed by the server 400. Although shown as internal to the server 400, it will be appreciated that the database may be accessed by the server over a network or via another wired or wireless connection. The server 400 may store desired or required information in the database 410 and may access the same to retrieve the information. The database 410 may include a number of tables adapted to store information relating to users.

The database 410 may be in communication with an object relational mapping (“ORM”) 420, also known as an object relational model or object-relational database management system. Although shown as internal to the server 400, it will be appreciated that the ORM may be accessed by the server over the network or via physical connection.

The ORM 420 may be in communication with a Universal Resource Indicator (“URI”) mapper 430 and a Rest API generator 440. The URI mapper 430 may map a URI into a pointer to an internal program, view, logic, or presentation of data within the system, based on one or more rules of a matching object specified in a collection of mapping objects. The matching object may be a regular expression. The URI mapper may be in communication with a web server 450.

The Rest API generator 440 may be in communication with a web server 450 as to send and/or receive data to and/or from client devices (e.g., user device 160 or provider device(s) 180) communicating with the server 400 (e.g., using HTTP and/or HTTPS). The Rest API generator 440 may prepare data stored in the database 410 for delivery to a client device or may prepare data received from a client device for storage in the database 410. The Rest API generator may be capable of translating between formats including, but not limited to JSON, XML and the like. The Rest API generator 440 may be capable of automatically generating URIs based upon data structures observed in the ORM 420 for access by client devices.

A web server 450 may be adapted to deliver web pages on request to users using the Hypertext Transfer Protocol (HTTP and/or HTTPS) or similar protocols. This allows for delivery of HTML documents and any additional content that may be included by a document, such as images, style sheets and scripts.

In one embodiment, a client device may employ a web browser or similar client application to engage in communication with the web server 450. For example, a client application may make a request for a specific resource using HTTP/HTTPS and the web server may respond with the content of that resource or an error message if unable to do so. The resource may be data or a file stored in a database 410. The web server 450 can receive content from a user, possibly using HTTP/HTTPS.

It will be apparent to one of ordinary skill in the art that, in certain embodiments, any of the functionality of the server may be incorporated into a client, and vice versa. Likewise, any functionality of a client application may be incorporated into a browser-based client, and such embodiments are intended to be fully within the scope of this disclosure. For example, a browser-based client application could be configured for offline work by adding local storage capability, and a native application could be distributed for various native platforms via a software layer that executes the browser-based program on the native platform.

The server 400 may comprise a monitoring application configured to receive activity information and to store the received information in the user's account. The server may keep track of the aggregate activity of the user, and prepare and/or send reports regarding the user's activity to the user and/or the user's provider (e.g., via a user device and/or a provider device). The reports may include, e.g., how often the user has moved outside of the predefined area, how long it takes (individual times and/or aggregates) to find the user after the user has moved outside of the predefined area, how often the user falls, how long it takes (individual times and/or aggregates) to provide help to the user after the user has fallen, the percentage of time that the user performs a certain activity (e.g., walking, running, sitting, standing, etc.), etc.

In certain embodiments, a user or provider may access the monitoring application running on the server via a user device 160 or a provider device 180. Generally, a user device or provider device may be any device capable of accessing the server, such as by running a client application or other software, like a web browser or web-browser-like application. Exemplary user or provider devices may include general purpose desktop computers, laptop computers, handheld devices, smartphones, smartwatches, personal digital assistants (PDA), and/or tablets.

Each user or provider device may have a client application running thereon, where the client application(s) may be adapted to communicate with the monitoring application running on the server. Such a configuration may allow users of client applications to input user information and/or interact with the monitoring application from any location that allows for access to the server.

Client applications may be adapted to present various user interfaces to users. Such user interfaces may be based on access privileges and/or information sent by the server, and may allow the user to send and receive data. Each client application may comprise HTML data, images, icons, and/or executable code. The executable code may be composed in JavaScript, ECMAscript, coffeescript, python, Ruby or any other programming languages suitable for execution within the client application, or translation into a client application executable form.

The client application(s) may be adapted to enter and/or update activity monitoring criteria for the user. The activity monitoring criteria may include, e.g., types of motion and/or orientation to be detected (e.g., being still, sitting, standing, lying down, walking, running, falling, etc.), frequency of sending activity information to the server, which activity transitions are to be sent to the server, etc. The applications may allow the user or provider to input user information, such as the name of the user and the ID of the user's motion detection device. The provider may also input notification preferences, such as the types of activities that require notification (e.g., moving outside the predefined area, falling or other types of motions/orientations, etc.) and a list of providers who should be notified when those activities occur.

The server may be implemented on any system with a processor that is capable of performing the functions described herein, including, but not limited to, server computers, desktop computers, laptop computers, handheld devices, tablet computers, smartphones, etc.

In one embodiment, communication between a client application and a monitoring application running on the server may involve the use of a translation and/or serialization module. A serialization module can convert an object from an in-memory representation to a serialized representation suitable for transmission via HTTP or another transport mechanism. For example, the serialization module may convert data from a native Python, Ruby, or Java in-memory representation into a JSON string for communication over the client-to-server transport protocol. After the JSON string is received by the receiver, a de-serialization module may convert the JSON string back into the native Python, Ruby, or Java in-memory representation for use by the client application or the patient monitoring and management application.

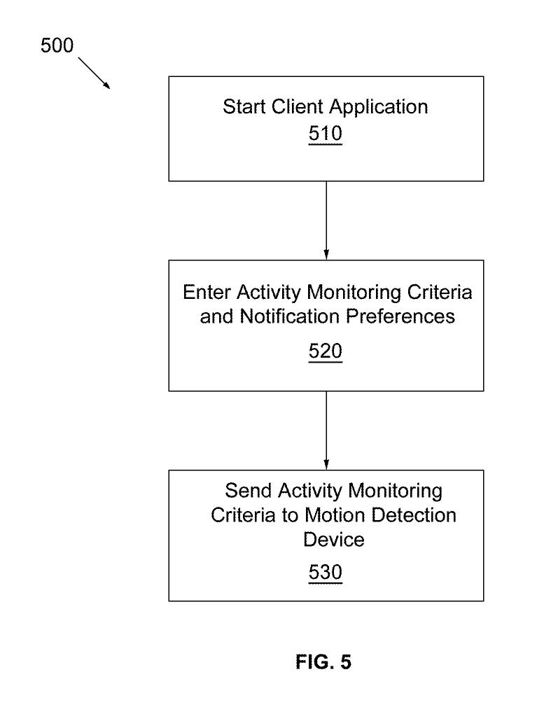

Referring to FIG. 5, a method 500 for setting up a notification in response to a particular activity is illustrated. In step 510, a client application is started on a client device (e.g., a user device or provider device).

In step 520, the activity monitoring criteria are entered. Activity monitoring criteria may include one or more of the following: the type of activity (e.g., being still, walking, running, falling, etc.), orientation (e.g., sitting, standing, lying down, etc.), and/or activity or orientation transition (e.g., being still to walking, sitting to standing, etc.) to monitor and/or detect; the frequency of sending activity information to the server during the activity, orientation, or transition; etc.

It will be appreciated that the activity monitoring criteria may be manually entered or automatically generated by the system based on user information.

Also in step 520, any desired notification preferences for the activity, orientation, or transition identified in the previous step are entered.

Entered notification information may include one or more of the following: the destination(s) (e.g., email address(es), phone number(s) (for SMS, etc.), motion detection devices, provider devices, etc.) of the notification; the type of notification (e.g., audio alert, visual alert, SMS, email, push notification of an app running on a provider device, etc.); notification content for SMS, email, and push notifications (e.g., a message that says “User A has fallen!”); the volume of an audio notification (including mute, or no sound); the number of times the audio notification sounds, the brightness and/or color of a visual notification; the number of LED flashes if the visual notification is an LED; the number of separate notification windows (e.g., times the notification will be presented); the time between notification windows; etc.

The user may also input a particular sequence of notifications, e.g., visual notification first, then an audio notification, and finally an SMS notification.

It will be appreciated that the notification preferences may be manually entered or automatically generated by the system based on user information. It will also be appreciated that some monitored activities may not require any notifications. For example, the total percentage of time the user spends in each orientation may be determined from continual monitoring of the user's orientation, but a provider may want to configure such monitoring to not generate any notifications.

In step 530, the activity monitoring criteria and notification preferences are sent to the motion detection device (e.g., directly, via user device in communication with the motion detection device, or via a gateway associated with the motion detection device). The motion detection device is identified by a unique ID, e.g., a MAC address or other ID. As discussed above, the motion detection device can then track the activity of the user and send activity information to the server.

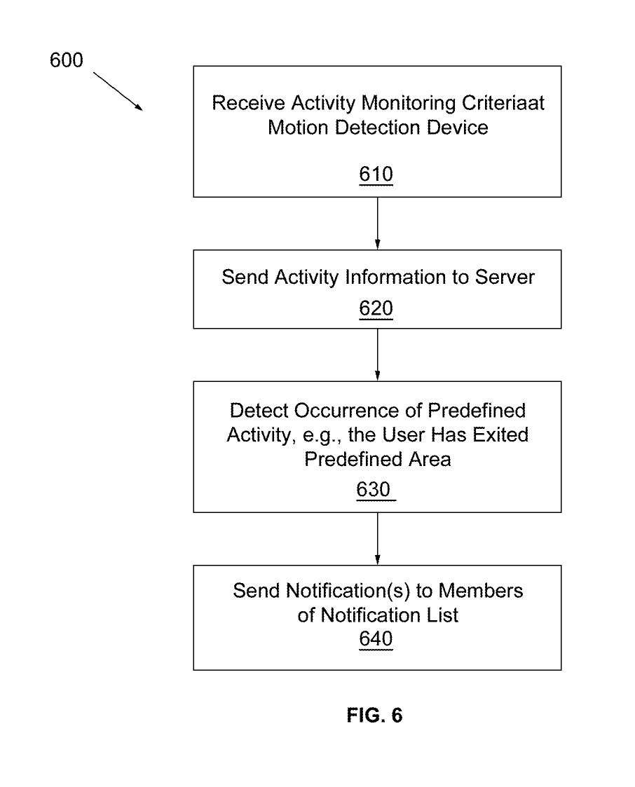

Referring to FIG. 6, a method 600 for tracking the activity of a user and notifying a provider is illustrated. In step 610, the motion detection device receives the activity monitoring criteria and notification preferences from the gateway 130 or server 140. In step 620, the motion detection device tracks the activity of the user in accordance with the activity monitoring criteria and sends the activity information to the server at the appropriate times. The server logs the activity information.

In step 630, the server detects that an activity, orientation, or transition, as defined by the activity monitoring criteria has occurred, e.g., that the user has fallen or that the user has exited the predefined area. In step 640, if a notification has been set up for the detected activity, orientation, or transition, the server sends notification(s) as indicated by the notification information.

Embodiments of the subject matter and the functional operations described in this specification can be implemented in one or more of the following: digital electronic circuitry; tangibly-embodied computer software or firmware; computer hardware, including the structures disclosed in this specification and their structural equivalents; and combinations thereof. Such embodiments can be implemented as one or more modules of computer program instructions encoded on a tangible non-transitory program carrier for execution by, or to control the operation of, data processing apparatus (i.e., one or more computer programs). Program instructions may be, alternatively or additionally, encoded on an artificially generated propagated signal (e.g., a machine-generated electrical, optical, or electromagnetic signal) that is generated to encode information for transmission to suitable receiver apparatus for execution by a data processing apparatus. And the computer storage medium can be one or more of: a machine-readable storage device, a machine-readable storage substrate, a random or serial access memory device, and combinations thereof.

As used herein, the term “data processing apparatus” comprises all kinds of apparatuses, devices, and machines for processing data, including but not limited to, a programmable processor, a computer, and/or multiple processors or computers. Exemplary apparatuses may include special purpose logic circuitry, such as a field programmable gate array (“FPGA”) and/or an application specific integrated circuit (“ASIC”). In addition to hardware, exemplary apparatuses may comprise code that creates an execution environment for the computer program (e.g., code that constitutes one or more of: processor firmware, a protocol stack, a database management system, an operating system, and a combination thereof).

The term “computer program” may also be referred to or described herein as a “program,” “software,” a “software application,” a “module,” a “software module,” a “script,” or simply as “code.” A computer program may be written in any form of programming language, including compiled or interpreted languages, or declarative or procedural languages, and it can be deployed in any form, including as a standalone program or as a module, component, subroutine, or other unit suitable for use in a computing environment. Such software may correspond to a file in a file system. A program can be stored in a portion of a file that holds other programs or data. For example, a program may include one or more scripts stored in a markup language document; in a single file dedicated to the program in question; or in multiple coordinated files (e.g., files that store one or more modules, sub programs, or portions of code). A computer program can be deployed and/or executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, such as but not limited to an FPGA and/or an ASIC.

Computers suitable for the execution of the one or more computer programs include, but are not limited to, general purpose microprocessors, special purpose microprocessors, and/or any other kind of central processing unit (“CPU”). Generally, CPU will receive instructions and data from a read only memory (“ROM”) and/or a random access memory (“RAM”). The essential elements of a computer are a CPU for performing or executing instructions and one or more memory devices for storing instructions and data. Generally, a computer will also include, or be operatively coupled to receive data from or transfer data to, or both, one or more mass storage devices for storing data (e.g., magnetic, magneto optical disks, and/or optical disks). However, a computer need not have such devices. Moreover, a computer may be embedded in another device, such as but not limited to, a mobile telephone, a personal digital assistant (“PDA”), a mobile audio or video player, a game console, a Global Positioning System (“GPS”) receiver, or a portable storage device (e.g., a universal serial bus (“USB”) flash drive).

Computer readable media suitable for storing computer program instructions and data include all forms of nonvolatile memory, media and memory devices. For example, computer readable media may include one or more of the following: semiconductor memory devices, such as erasable programmable read-only memory (“EPROM”), electrically erasable programmable read-only memory (“EEPROM”) and/or and flash memory devices; magnetic disks, such as internal hard disks or removable disks; magneto optical disks; and/or CD ROM and DVD-ROM disks. The processor and the memory can be supplemented by, or incorporated in, special purpose logic circuitry.

To provide for interaction with a user, embodiments may be implemented on a computer having any type of display device for displaying information to a user. Exemplary display devices include, but are not limited to one or more of: projectors, cathode ray tube (“CRT”) monitors, liquid crystal displays (“LCD”), light-emitting diode (“LED”) monitors and/or organic light-emitting diode (“OLED”) monitors. The computer may further comprise one or more input devices by which the user can provide input to the computer. Input devices may comprise one or more of: keyboards, a pointing device (e.g., a mouse or a trackball). Input from the user can be received in any form, including acoustic, speech, or tactile input. Moreover, feedback may be provided to the user via any form of sensory feedback (e.g., visual feedback, auditory feedback, or tactile feedback). A computer can interact with a user by sending documents to and receiving documents from a device that is used by the user (e.g., by sending web pages to a web browser on a user's client device in response to requests received from the web browser).

Embodiments of the subject matter described in this specification can be implemented in a computing system that includes one or more of the following components: a backend component (e.g., a data server); a middleware component (e.g., an application server); a frontend component (e.g., a client computer having a graphical user interface (“GUI”) and/or a web browser through which a user can interact with an implementation of the subject matter described in this specification); and/or combinations thereof. The components of the system can be interconnected by any form or medium of digital data communication, such as but not limited to, a communication network. Non-limiting examples of communication networks include a local area network (“LAN”) and a wide area network (“WAN”), e.g., the Internet.

The computing system may include clients and/or servers. The client and server may be remote from each other and interact through a communication network. The relationship of client and server arises by virtue of computer programs running on the respective computers and having a client-server relationship to each other.

Various embodiments are described in this specification, with reference to the detailed discussed above, the accompanying drawings, and the claims. Numerous specific details are described to provide a thorough understanding of various embodiments. However, in certain instances, well-known or conventional details are not described in order to provide a concise discussion. The figures are not necessarily to scale, and some features may be exaggerated or minimized to show details of particular components. Therefore, specific structural and functional details disclosed herein are not to be interpreted as limiting, but merely as a basis for the claims and as a representative basis for teaching one skilled in the art to variously employ the embodiments.

The embodiments described and claimed herein and drawings are illustrative and are not to be construed as limiting the embodiments. The subject matter of this specification is not to be limited in scope by the specific examples, as these examples are intended as illustrations of several aspects of the embodiments. Any equivalent examples are intended to be within the scope of the specification. Indeed, various modifications of the disclosed embodiments in addition to those shown and described herein will become apparent to those skilled in the art, and such modifications are also intended to fall within the scope of the appended claims.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

All references including patents, patent applications and publications cited herein are incorporated herein by reference in their entirety and for all purposes to the same extent as if each individual publication or patent or patent application was specifically and individually indicated to be incorporated by reference in its entirety for all purposes.

What is claimed is:

1. A device that attaches to a user's body, comprising
a location detector;
a motion detector;
an orientation detector;
a communication module for transmitting user activity information to a server based on activity monitoring criteria, wherein the user activity information causes the server to transmit periodic notifications to a provider;
a proximity detector for detecting that the device has been removed from the user's body; and
means for causing the server to stop transmitting the notifications.

2. The device of claim 1, wherein the user activity information comprises the user's location and the notifications are transmitted based on the user having left a predefined geographic area.

3. The device of claim 1, wherein the user activity information comprises the user's motion, and the notifications are transmitted based on the user falling or haven fallen.

4. The device of claim 1, wherein the proximity detector comprises an infrared detector.

5. The device of claim 1, wherein the communication module transmits the user activity information to a gateway and the gateway transmits the user activity information to the server.

6. A method for notifying a provider that a user possessing a motion detection device needs assistance, comprising:
receiving, by a server, activity information regarding the user from the motion detection device, the activity information based on activity monitoring criteria previously transmitted to the motion detection device;
determining, by the server, that the user needs assistance based on the received activity information; and
sending, by the server, periodic notifications to the provider regarding the assistance needs of the user;
wherein the motion detection device comprises a button that may be pressed by the provider causing the server to stop sending the notifications.

7. The method of claim 6, wherein the user activity information comprises the user's location and the notifications are transmitted based on the user having left a predefined geographic area.

8. The method of claim 6, wherein the user activity information comprises the user's motion, and the notifications are transmitted based on the user falling or haven fallen.

9. The method of claim 6, wherein the motion detection device comprises a proximity detector for detecting that the device has been removed from the user's body.

10. The method of claim 9, wherein the proximity detector comprises an infrared sensor.

11. The method of claim 6, wherein the server receives the user activity information from a gateway, wherein the gateway had previously received the user activity information from the motion detection device.

12. A system comprising one or more processors and one or more storage devices storing instructions that when executed by the one or more processors cause the one or more processors to perform operations comprising:
receiving, from a motion detection device, activity information regarding a user possessing the motion detection device, the activity information based on activity monitoring criteria previously transmitted by the system to the motion detection device;
determining that the user needs assistance based on the received activity information; and
sending periodic notifications to a provider regarding the assistance needs of the user;
wherein the motion detection device comprises a button that may be pressed by the provider causing the server to stop sending the notifications.

13. The system of claim 12, wherein the user activity information comprises the user's location and the notifications are transmitted based on the user having left a predefined geographic area.

14. The system of claim 12, wherein the user activity information comprises the user's motion, and the notifications are transmitted based on the user falling or haven fallen.

15. The system of claim 12, wherein the motion detection device comprises a proximity detector for detecting that the device has been removed from the user's body.

16. The system of claim 15, wherein the proximity detector comprises an infrared sensor.

17. The system of claim 12, wherein the system receives the user activity information from a gateway, wherein the gateway had previously received the user activity information from the motion detection device.
